(19) United States
(12) Patent Application Publication
(10) Pub. No.: US 2021/0346538 A1
(43) Pub. Date: Nov. 11, 2021

(54) CURRENCY SANITIZER

(71) Applicant: Perry W. Haddon, Prescott, AZ (US)

(72) Inventor: Perry W. Haddon, Prescott, AZ (US)

(21) Appl. No.: 16/871,673

(22) Filed: May 11, 2020

(51) Int. Cl. A61L 2/10; B08B 7/00; A61L 2/26

(52) U.S. Cl. CPC A61L 2/10; A61L 2202/11; A61L 2/26; B08B 7/0057

(57) ABSTRACT

One of the most common surfaces handled by a multitude of persons, without intermediate cleansing, is money. Viruses, bacteria, germs, and any other infectious elements that may cause disease, are generally transmitted via contact with a person who is infected or has recently touched a person or surface which has been thus contaminated. This invention fulfills society's need for money handlers to sanitize previously contaminated currency before they themselves become infected. The invented device may also be used by the money handler to sanitize currency before making change and giving it to another customer. This cycle may be interrupted and thereby reducing the spread of communicable diseases.

### BACKGROUND OF THE INVENTION

### Field of the Invention

The field of the invention is decontamination of ever present virus, bacteria, germs, or any infectious elements, found on commonly handled currency, playing cards, credit cards, coupons, or identification (OBJECT ITEMS) that can spread from person to person without the knowledge or intention of the handler.

### Prior Art

Pub. No.: US 2020 0101183 AI Dijkstra et al. bears only minor similarity in that it employs UV light to decontaminate surfaces which has been done for nearly one half a century, but does not address the instant application's utility and specific application.

No other prior art or invention could be found that meets the need for small space consumption on retail counters or expresses the utility purpose of sanitizing currency before money handlers exposure or prior to the redistribution of the currency.

### BRIEF SUMMARY

### Object of the Invention

Viruses, bacteria, germs, and/or other infectious elements that can cause or convey disease are omnipresent on circulated currency, credit cards, playing card, or other OBJECT ITEMS. All too frequently, knowingly or unknowingly, an infected person or carrier hands, money to a store teller without a thought of potential transmission of infection. Recipients, who are regularly placed in this danger, then recirculates the currency in the form of change given to another the customer or bank teller. This invention attempts brake that cycle and mitigate the danger by sanitizing currency as it is exchanged and recirculated.

An incidental side effect is repeated exposure of UV light, especially UVC light, which in seconds can cause skin burns. The instant device is shielded from escaping UV light to avoid personal exposure.

### BRIEF DESCRIPTION OF DRAWINGS

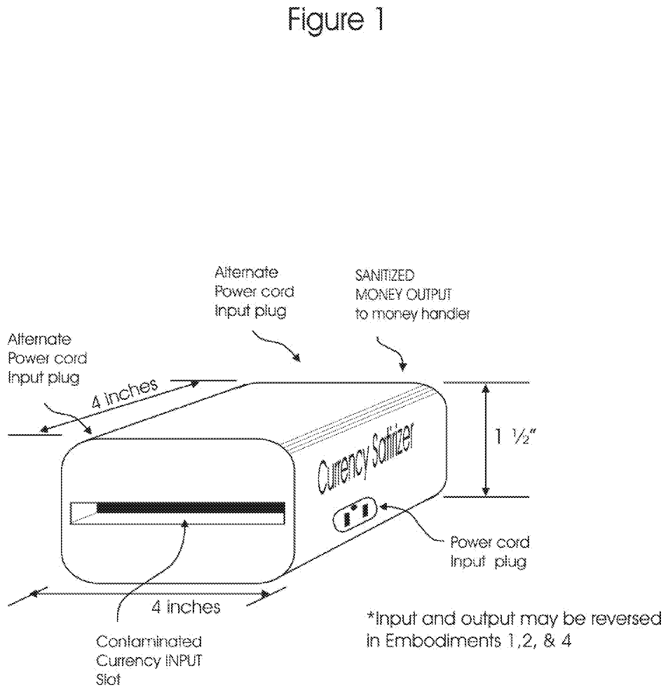

The pictorial view of the CURRENCY SANITIZER is illustrated in FIG. 1.

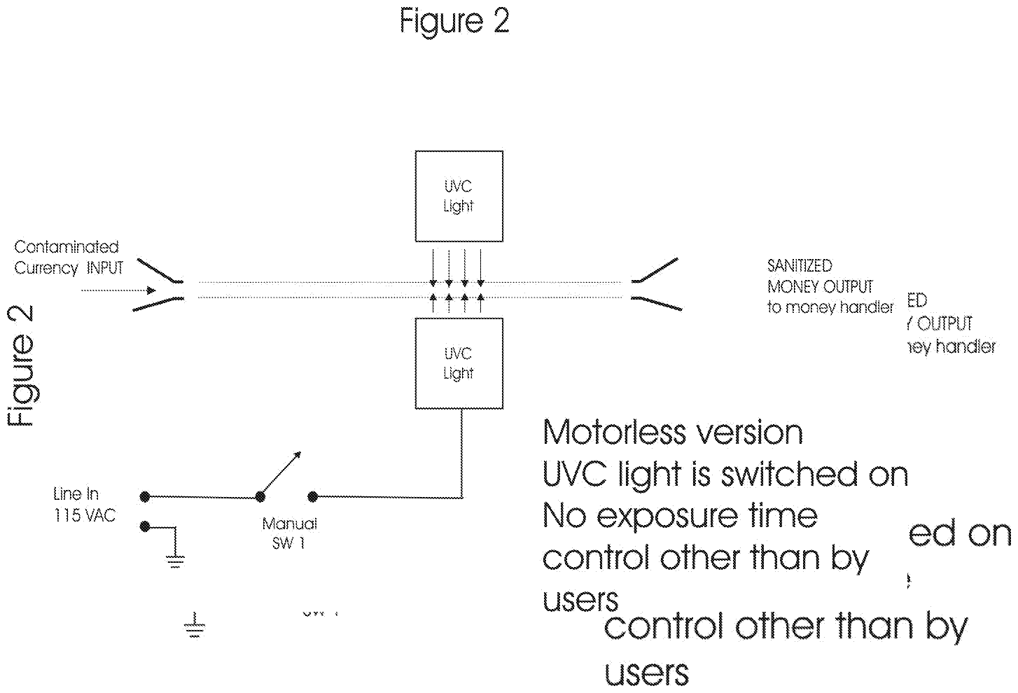

Block schematic diagram of the CURRENCY SANITIZER embodiments 1-4 are illustrated in FIGS. 2-5.

### BRIEF DESCRIPTION OF THE DRAWING ELEMENTS

FIG. 1 CURRENCY SANITIZER pictorial view of the device

FIGS. 2-5 Are the embodiments 1-4 of the CURRENCY SANITIZER are representative of the overall patent in block schematic diagram of the embodiments.

### DETAILED DESCRIPTION OF THE EMBODIMENTS 1-4 AS FOUND IN DRAWINGS FIGS. 1-5

The embodiments 1-4 (FIGS. 1-5) of the CURRENCY SANITIZER are representative of the overall patent and are made to an electrical and mechanical size that can accommodate the limited counter space available on the retailer's counter (FIG. 1). It is electronically simple enough to be economically manufactured for ubiquitous use (FIG. 2) and is convenient for both the customer and money handler (conveyor and recipient) of currency (FIG. 1). The device is intended to become intuitive for people to look for the device as they approach a public pay station to sanitize the currency as they hand, indirectly through the device, their payment to the recipient and/or in reverse, as change is handed back to the customer.

The device may be scaled up from FIG. 1 to accommodate higher volumes of currency and where space is less of a problem.

FIG. 1 Is the CURRENCY SANITIZER pictorial view. The picture is common to the device in all its embodiments. The OBJECT ITEMS are inserted in one end of the unit where both sides are exposed to decontaminating UV light and exits in a cleaner, more sanitary state at the other.

FIG. 2 is a block schematic diagram of embodiment 1 and is made in the most simplistic-rudimentary form as to be economically mass produced and preform its intended purpose of decontaminating the OBJECT ITEMS being conveyed to the teller or in the reverse, decontaminated change may be conveyed back to the customer.

The OBJECT ITEMS pass in direct proximity to the source lights to avoid inverse squared exposure losses due to distance from the sources lights. While the light intensity may be effective when exposed for an adequate duration, the timed exposure is significantly reduced by its close proximity.

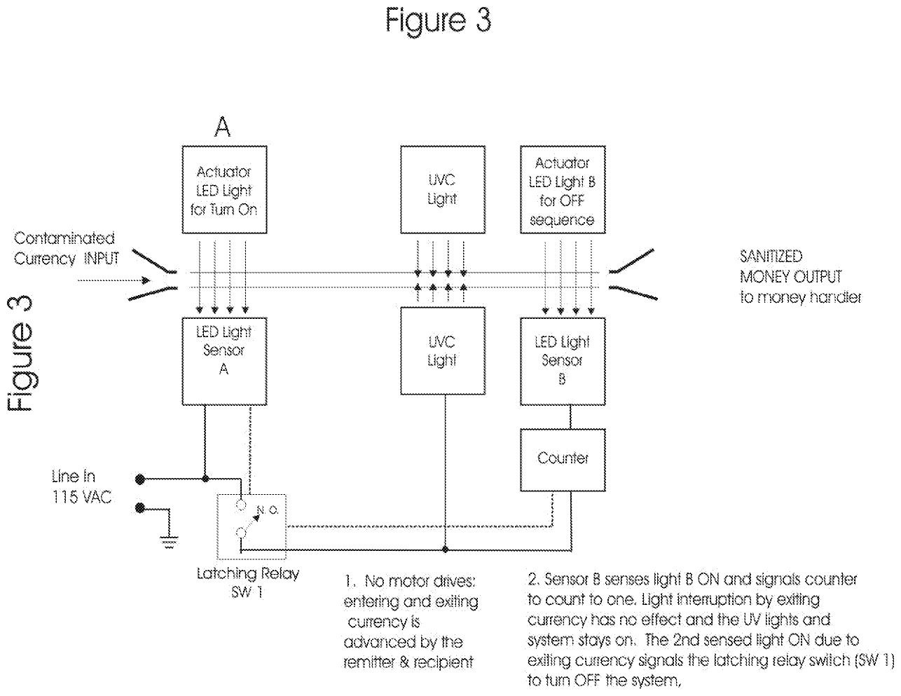

FIG. 3 is a block schematic diagram of embodiment 2 and is made to serve the purposes of Embodiment 1 and is equipped to be more convenient to the customer and money handler (conveyor and recipient) in that the device is automatically turned on by the insertion of OBJECT ITEMS to be decontaminated (SW 1). The off function is actuated by the recipient when the item is retrieved and Actuator LED light B is again present on Sensor B which again activates Latching relay switch SW 1 and turns off the device. The device may be fed and actuated from either end for the convenience of both conveyor and recipient.

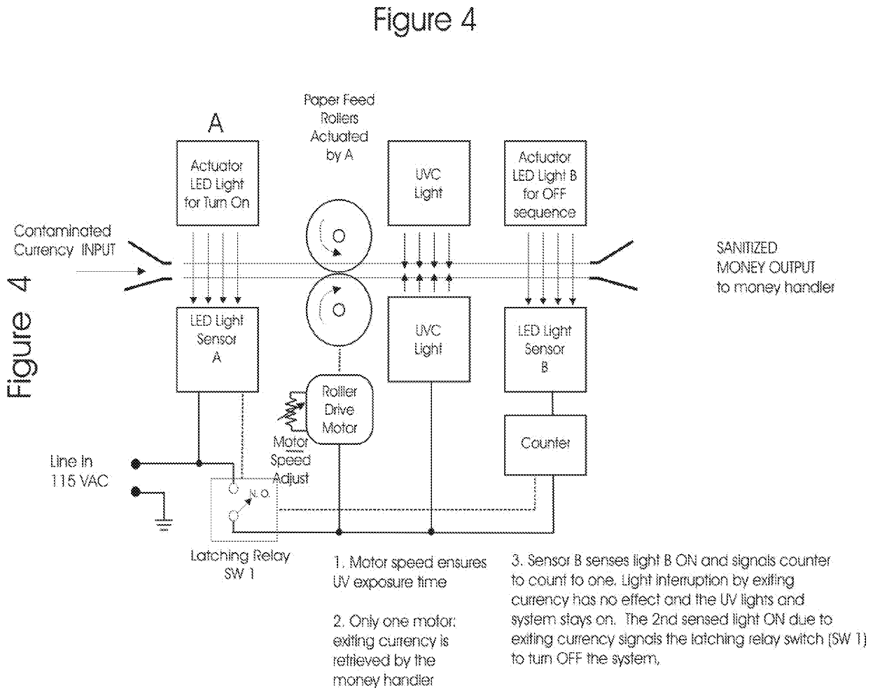

FIG. 4 is a block schematic diagram of embodiment 3 which is made to serve the purposes of Embodiment 1 and 2 and is made to serve its main purpose of decontamination by employing a motor drive which ensures and regulates the exposure time of the OBJECT ITEMS. The motor is field-set to a speed consistent with CDC or other laboratory recommendations which may vary according to anticipated levels and types of contamination, taking into consideration their inherent severity, their ease in neutralizing, and communicable character.

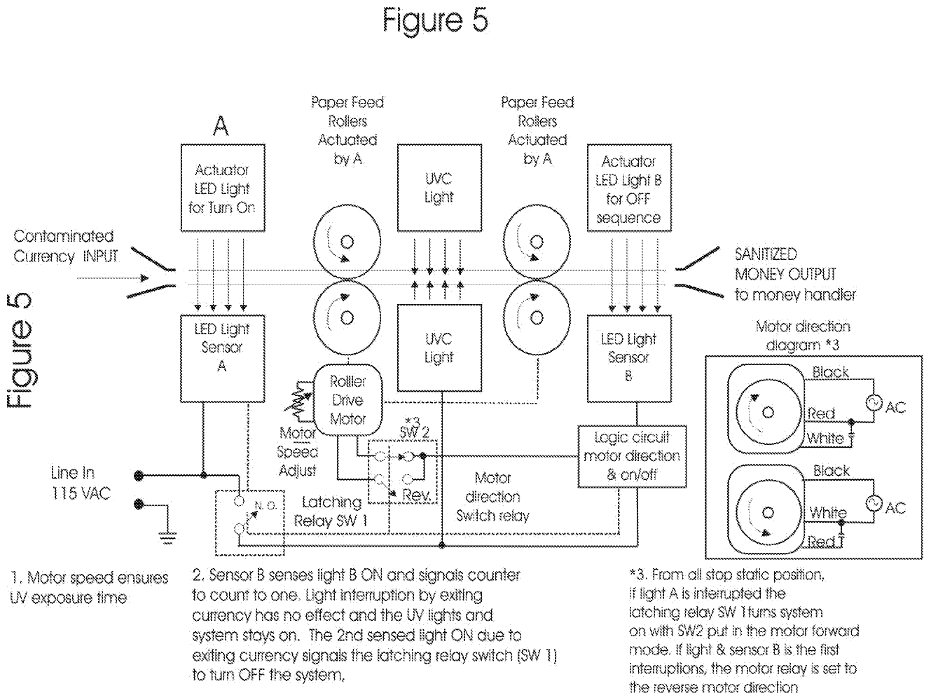

FIG. 5 is a block schematic diagram of embodiment 4 which is made to serve the purposes of Embodiment 1, 2 and 3 and is made to be more convenient to the customer and money handler (conveyor and recipient) in that the motors are reversible and may work in a forward or reverse mode depending on which end the OBJECT ITEMS are inserted.

When the OBJECT ITEMS interrupt the exiting Light sensor, A or B depending on the direction of the OBJECT ITEM travel, the rollers and UV lamps continue to stay on until the object exits and exiting Actuator LED light (A or B) is again present upon its corresponding Sensor, and the latching-relay (SW1) again toggles and returns it to its open position thereby turning off the unit. This action serves to preserve the UV lamp bulbs that have a limited number of lamp on hours as well as serves the users who do not have to turn off the device.

In the all embodiments 1-4 (FIGS. 2-5), money is inserted into the device one bill at a time for decontamination.

The Money Sanitizer now stands ready for the next transaction.

Other actuating procedures may be employed for this purpose of turning the device ON or OFF such as, but not limited to, manual switches (as in embodiment 1), manual micro switches tripped by the OBJECT ITEMS, or proximity switches.

Note: The term Ultra Violet light (UV) is used in the generic and includes UVC light, which is more effective in its disinfecting characteristics and the term “bulb” may be interchanged with LED.

The drive circuits are very similar to those used by ATM machines, cash change makers, and candy or soda pop dispensers and are not included in the CLAIMS.

I claim as follows:

1. A device that conveniently decontaminates currency, identification cards, playing cards, other papers, or credit cards (OBJECT ITEMS) that are routinely passed from person to person.

2. A method wherein decontamination of both sides of an item are disinfected by directing the item between two UV lights at one time.

3. The rate of speed at which the object passes between UV lights (exposure time) can be field-set by regulating a drive motor speed depending upon current contamination threats.

4. The intensity of the UV light may be varied by rheostat and/or by utilizing different intensity UV sources and can be field-set depending upon current contamination threats.

5. A method of placing flat objects (money, credit cards or other objects) in direct proximity to a UV light in order to gain maximum exposure to the source light thus minimizing inverse squared exposure losses due to the distance.

6. A design of a device that may be made small enough to be suitable for use on limited retail counter space.

7. An invention in its simplest form (FIG. 2) wherein high intensity UV lights and close object proximity allows for decontamination in short enough exposure times to alleviate concerns of under-exposure by hand fed objects while lessening the overall costs of including expensive exposure controls and/or on/off circuitry, thus make a device affordable for wide spread use.

8. Use of transparent glass or plastic to separate the object paper or plastic from the potentially hot UV source and to simultaneously allow for close proximity.

9. The feed can be personal (one item at a time) or via an auto feeding which can accommodate large volume handlers of currency for decontaminations.

10. The device can be automatically turned on and off between uses to preserve the UV lamps' life which is rated by the manufacturer in predictable hours of use.

11. The device may be fed from either direction (forward or backward) to disinfect items from the consumer's direction or from the money handler's direction utilizing reversible motors or hand pushed items.

12. The device is shielded from the users in order to eliminate out-of-box UV exposure and potential burns by prolonged and/or repeated use.

13. Multiple power input jacks provide an increase in flexibility where installation area is very limited on counter tops.
